(19) United States
(12) Patent Application Publication
(10) Pub. No.: US 2014/0163325 A1
(43) Pub. Date: Jun. 12, 2014

(54) MOUTHPIECE

(71) Applicant: Gloria Hartston, Ontario, CA (US)

(72) Inventor: Gloria Hartston, Ontario, CA (US)

(21) Appl. No.: 13/711,462

(22) Filed: Dec. 11, 2012

(51) Int. Cl. A61B 13/00; A61M 1/00; A62B 9/06

(52) U.S. Cl. CPC A61B 13/00; A62B 9/06; A61M 1/0066
USPC 600/205; 264/16

(57) ABSTRACT

The disclosure is of a device used for dislodging an object occluding the upper trachea of a human choking on that object, the method of use, and the method of manufacturing the device itself. The device is a mouthpiece comprising three main parts, namely a mouth cover, a hollow tongue depressor and an attached nose clamp. The mouth cover provides a nozzle in fluid communication with the hollow tongue depressor, and the nozzle is adapted to be connected to a vacuum generator. The hollow tongue depressor is adapted to be inserted into the choking human's mouth and to deliver a vacuum differential generated by the vacuum generator and transmitted through the nozzle to the mouth cavity of the human through the hollow tongue depressor. The mouth cover is used to create a closed system by sealing the mouth and lips of the choking human and thereby create the pressure differential in the upper trachea of the choking human when a vacuum is generated. The nose clamp is used to complete the closed system. The method of using the device is disclosed, as is the method of manufacturing the device.

### CROSS REFERENCE TO RELATED APPLICATIONS

This patent application claims priority from U.S. provisional application No. 61/567,008

### BACKGROUND OF THE INVENTION

1. Field of the Invention

The field of the invention includes a mouthpiece adapted to enable creation of a pressure differential in a human's trachea when the mouthpiece is used with a vacuum source.

2. Description of Related Art

An expired patent U.S. Pat. No. 3,946,736 to Neward discloses a suction device that dislodges foreign objects from above the epiglottis of a human.

### BRIEF SUMMARY OF THE INVENTION

This device is a specially adapted mouthpiece comprising a mouth cover, a hollow tongue depressor and a nose clamp. The mouth cover is shaped to sealingly fit over and to completely cover the lips and mouth of a human. The mouth cover defines a nozzle opening that is adapted to sealingly mate with and to be in fluid connection with a vacuum source that has the capacity to generate from about 5 to about 50 inches of mercury of pressure. Vacuum generated by such a source is fluidly transmitted through the mouth cover and into the human's mouth cavity through a hollow tongue depressor. The depressor projects away from the mouth cover, is fluidly connected to the nozzle and defines several holes through which the pressure differential created by the vacuum source can be delivered to the mouth cavity and, therethrough, to the throat of a human. The nares of the human's nose are clamped shut by the nose clamp to create a closed system. When a human is choking because a foreign object is stuck in the human's trachea, the assembled device is used with a vacuum source to vacuum the object out of its occluding position within the throat or upper trachea and relieve the choking

The steps for using this device include: inserting the mouthpiece into the choking human's mouth, depressing the human's tongue down away from the palate; sealing the human's nose with the nose clamp; applying a vacuum through the mouthpiece to create a pressure differential across an occluding object in the human's trachea, thereby dislodging the object. The mouthpiece forms a seal around the lips of the choking human's mouth. The nose clamp is placed on the nares of the human's nose. The mouthpiece is fluidly connected to a vacuum source capable of generating from about 5 to about 50 inches of mercury of pressure. When the differential pressure across the foreign object in the trachea is sufficient, the object is dislodged from the trachea and urged toward the end of the tongue depressor where it may be removed manually. The device is then removed from the formerly choking human.

### BRIEF DESCRIPTION OF THE DRAWINGS

FIG. 1 is a side and top perspective view of one embodiment of the device of this application in use on a human with an object lodged in the trachea.

FIG. 2 is a rear perspective view of the vacuum generator illustrated in FIG. 1.

FIG. 3 is a front perspective view of the combination mouthpiece and nose clamp illustrated in FIG. 1.

FIG. 4 is a rear perspective view of the combination mouthpiece and nose clamp illustrated in FIG. 1.

FIG. 5 is a side view of a sagittal section of a human nose, mouth and chin and the mouthpiece and nose claim illustrated in FIG. 1.

### DETAILED DESCRIPTION

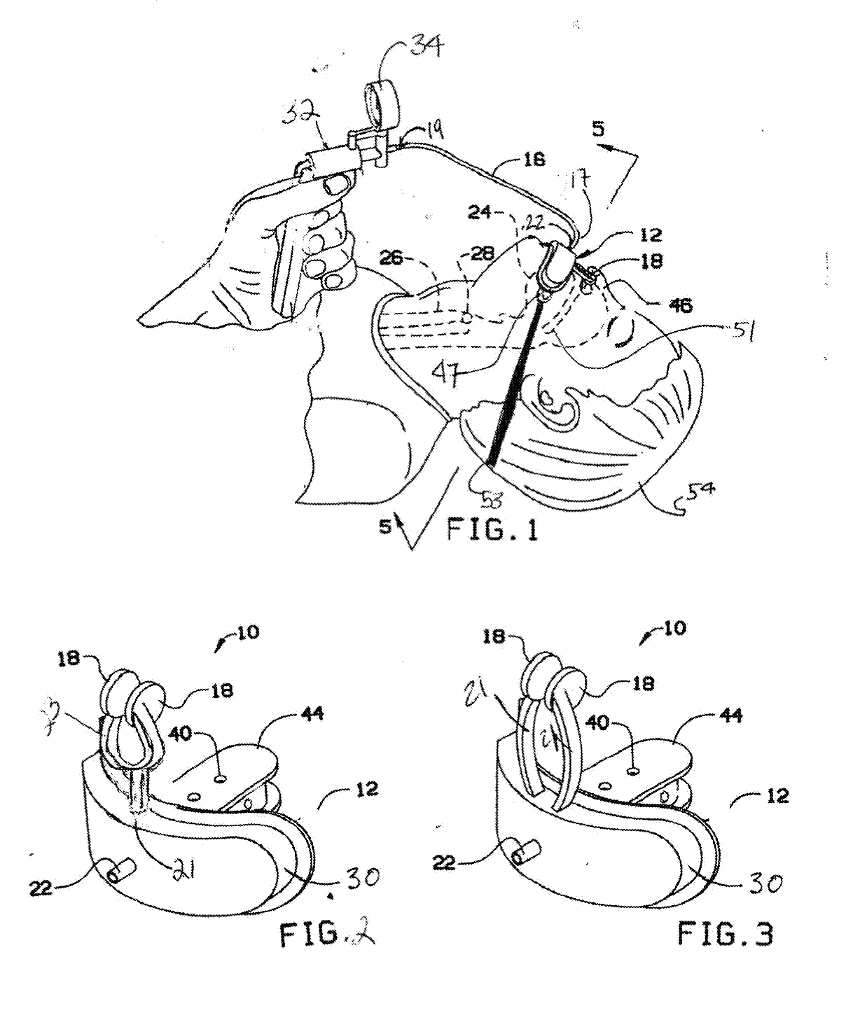

Please refer to FIG. 1. This drawing illustrates a device according to the disclosure 10 in use on a human 54 whose upper trachea 26 is occluded by a foreign object 28. The device 10 includes a curved mouth cover 12 adapted to sealingly fit over the human's 54 mouth. The mouth cover 10 includes a nozzle opening 22 adapted to be fluidly and sealingly connected to a vacuum source. In one embodiment, the vacuum source may be a vacuum tube 16 which is itself fluidly attached to a vacuum pump 32.

The vacuum generator 32 may be a hand pump or a motorized pump (not shown) or any vacuum device capable of producing a pressure differential of at least 5 to 50 inches of mercury. The generator 32 may include a pressure indicator 34 to indicate to the caregiver operating the vacuum generator 32 the amount of pressure being created. Should there be no pressure differential created, it is unlikely that the choking human 54 being treated using the device 10 has an occluded trachea.

Referring to FIGS. 1 and 2, a device according to the disclosure 10 is shown in detail and includes a vacuum delivery hollow tongue depressor 44. The tongue depressor 44 is in fluid communication with the vacuum nozzle 22. The depressor 44 has vacuum holes 40 along its surfaces through which holes 40 the vacuum is delivered to the human's mouth cavity 24. The mouth cavity 24 is connected to the trachea 26 in which a foreign object 28 is lodged. The mouthpiece 10 may be secured in place through the use of a restraining band 53 threaded through loops 47 or projections (not shown) on the mouth cover 12.

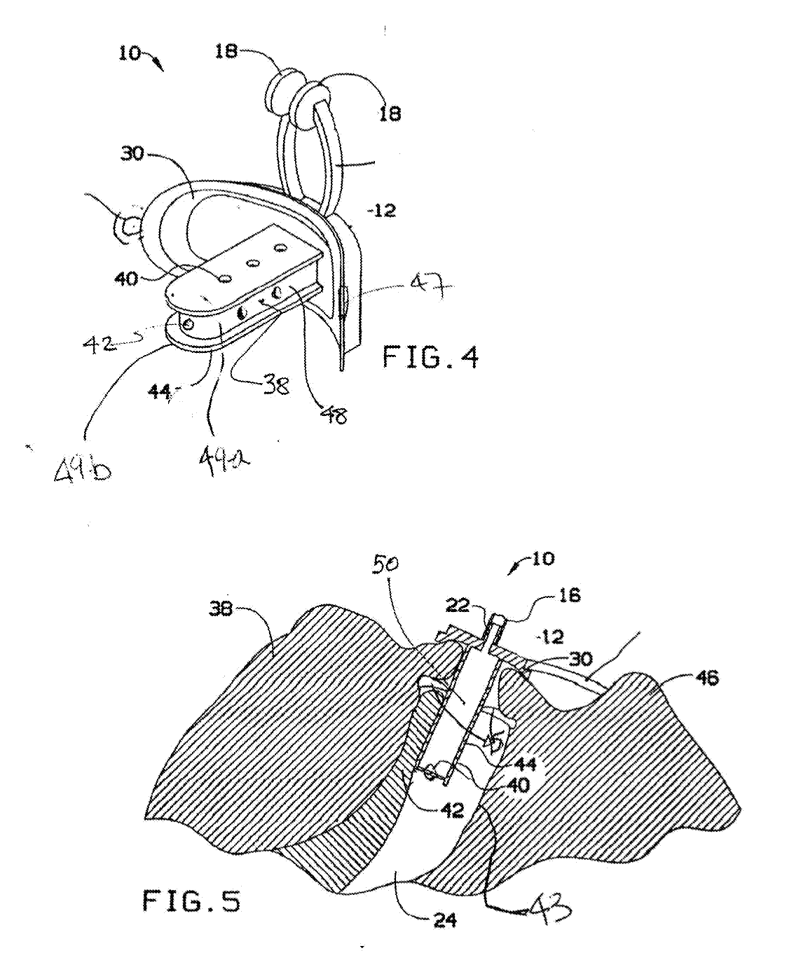

Referring again to FIG. 2, the device 10 may include a U-shaped nose clamp 23 comprising nares sections 18 and a living hinge 21. Referring to FIG. 3, the device may alternately comprise a two part nose clamp, each of which parts comprises nares sections 18 and a living hinge 21.

Referring to FIG. 4, the mouth cover 12 may comprise a lip cavity 30 on its inner surface to accommodate human lips and so sealingly cover the mouth. When the nares of a human's nose 46 are clamped shut, either by the U-shaped nose clamp 23 or by the pair of separately attached clamps 21 and the mouth cover 12 is sealingly fit over the human's lips, a closed system is created in which a vacuum source may create a pressure differential.

Still referring to FIG. 4, a lip cavity 30 adapted to accommodate the lips of a human is defined by the curved mouth cover 12. A marginal flange 12 extends from the perimeter of the lip cavity 30 and is adapted to create a sealing edge when placed in contact with a human face about the mouth. In one embodiment, the tongue depressor 44 comprises a pair of spaced apart essentially flat, blade elements 49a and 49b separated by a spacing wall 48. Vacuum holes 40 in each of these elements 40 effect transmission of vacuum delivered from the mouthpiece 10 to the mouth cavity 24.

As seen in FIG. 5, the tongue depressor 44 projects into a human mouth cavity 24, depressing the human's tongue 42 and is spaced apart from the human's palate 43. The human's teeth 45 assist in positioning the tongue depressor 44. Holes 40 allow the pressure differential created by the vacuum source to be transmitted into the mouth cavity 24 and the upper trachea 26 when the nares are clamped shut by the nose clamp 23.

The entire device 10 can be made from a single injection mold adapted for use in injection molding of polypropylene. While other thermoplastics could be used in manufacturing a device 10 of the disclosure, polypropylene presents many advantages over other plastics: It has a relatively low melting point, making it more economical to heat to melting than polystyrene; it is flexible and unlike polystyrene, can form a “living hinge” for both the U-shaped nose clamp 23 and for a two part nose clamp 21. Polypropylene is also relatively inexpensive when compared with other plastics including polystyrene, and polypropylene is resistant to stomach acids which may be regurgitated onto the device 10 during use. Disposal after a single use of the device 10 is recommended.

It is important to provide the noseclamp 21, 23 as a part of the device 10 since, without a means for forming a closed system, vacuum generated and communicated to a human's mouth chamber is dissipated. It is also important that the nose clamp 21, 23 remain attached to the device so that it is not misplaced or lost in the process of sealing the choking human's mouth and nose.

Use of a device 10 dimensioned according to this disclosure is used as follows: The mouthpiece 10 is fluidly attached to a vacuum generator 32 capable of generating from about 5 inches to about 50 inches of mercury of pressure; the mouthpiece is inserted into the mouth of a choking human 54 with the tongue depressor 44 positioned within the human's mouth cavity, depressing the human's tongue down away from the palate, and the vacuum nozzle 22 positioned outside of the human's mouth cavity 24; the mouth cover 12 is sealingly positioned over the choking human's mouth and lips; the choking human's nose 46 is sealed using the nose clamp 21, 23; a pressure differential across the object upon which the human is choking 28 is created when the vacuum generator 32 is activated. When the differential pressure across the foreign object 28 in the trachea is sufficient, the object 28 is dislodged from the trachea and urged toward the end of the tongue depressor 44 where it may be removed manually. The device 10 is then removed from the formerly choking human 54.

A method of manufacturing the mouthpiece 10 includes the following steps: A mold is provided that is adapted for use in thermoplastic injection molding and that is adapted to create a device 10 of this disclosure through a single molding process. Nurdles of thermoplastic are introduced into the hopper of an injection molding machine, the mold is positioned, and the molding machine is activated. Thermoplastic nurdles are fed from the hopper into a heating section of the machine, and then when melted, the thermoplastic is injected into the mold. Once the mold is sufficiently cooled to enable the injected thermoplastic to harden, the mold is removed and a device 10 according to this disclosure is created and available for use. Other methods of manufacture known in the trade may also be used as long as there is but one mold and one “pass” through the molding process required.

Although the present invention has been described in considerable detail with reference to certain preferred versions thereof, other versions are possible. Therefore, the spirit and scope if the appended claims should not be limited to the description of the preferred versions contained herein.

All the features disclosed in this specification (including any accompanying claims, abstract and drawings) may be replaced by alternative features servicing the same, equivalent or similar purpose, unless expressly stated otherwise, Thus unless expressly stated otherwise, each feature disclosed is one example only of a generic series of equivalent or similar features.

I claim:

1. A device comprising:
a. a curved mouth cover adapted to sealingly fit over a human's mouth and lips;
b. the mouth cover defining a nozzle opening adapted to be sealingly connected to and in fluid communication with a vacuum generator;
c. the mouth cover further including a vacuum delivery hollow tongue depressor having at least one wall that defines at least one hole, the depressor integrally connected to the mouth cover and in fluid communication with the vacuum tube nozzle, the tongue depressor projecting away from the cover; and
d. a nose clamp having at least one living hinge and adapted to clamp the nares of a human's nose shut.

2. A device according to claim 1 wherein the vacuum source is a hand pump and is capable of generating a vacuum of 5 to 50 inches of mercury.

3. A device according to claim 1 wherein the combination mouth cover, nose clamp and hollow tongue depressor is constructed of injection molded polypropylene.

4. A device according to claim 1 wherein the mouth cover is adapted to accommodate a pair of human lips and to sealingly fit over the human's lips and mouth.

5. A device according to claim 1 wherein the mouth cover includes a vacuum sealing marginal flange.

6. A device according to claim 1 wherein the tongue depressor comprises a hollow tongue depressor in fluid communication with the vacuum generator.

7. A device according to claim 1 wherein the at least one wall of the tongue depressor defines at least one hole.

8. A device according to claim 1 wherein the vacuum delivery hollow tongue depressor comprises a pair of spaced apart blade elements, the first of the pair of blade elements adapted to flatten a human tongue and the second of the pair of blade elements adapted to be spaced apart from a human palate, the blades being spaced apart from each other by a spacing wall.

9. A device according to claim 1 wherein the vacuum delivery hollow tube has a proximal end and a distal end, the proximal end is fluidly connected to the nozzle opening, and the at least one wall defines at least one hole.

10. A device according to claim 1 wherein the nose clamp comprises a substantially U-shaped clamp adapted to clamp and sealingly close the nares of a human's nose, which clamp is itself flexible and which clamp is flexibly connected to the mouthpiece through a living hinge.

11. A device according to claim 1 wherein the nose clamp provided comprises a pair of living hinges connected to the mouthpiece and adapted to clamp and sealingly close the nares of a human's nose.

12. A device according to claim 1 wherein the mouth cover defines at least a pair of holes through which a retaining band is threaded, the retaining band adapted to fit behind the head of a human when the mouthpiece is inserted into the human's mouth and to retain the mouthpiece therein.

13. A method of removing an object from an occluded human trachea comprising the steps of;
a. providing a vacuum source adapted to create a pressure differential across a vacuumed object from about 5 inches of mercury to about 50 inches of mercury, the source providing a vacuum port;
b. providing an injection molded polypropylene mouthpiece comprising a sealing mouth cover, a nares clamping nose clamp, a nozzle adapted to sealing mate with the vacuum source, and a hollow vacuum delivery tongue depressor comprising at least one wall that defines air holes, the tongue depressor itself fluidly connected to the vacuum port;
c. fluidly and sealingly attaching the mouthpiece to the vacuum generator;
d. inserting the mouthpiece into the mouth of a human choking on an object stuck in the human's upper trachea, sealing the mouth cover around the lips and mouth of the human, clamping the nose clamp onto the nares of the choking human and securing the mouthpiece to the human's head;
e. providing a vacuum of about 5 to about 50 inches of mercury from the vacuum source and creating a pressure differential of the same across the object occluding the human's upper trachea until the object is dislodged therefrom.

14. A method according to claim 13 wherein the vacuum generator provided comprises a hand pump.

15. A method according to claim 13 wherein the vacuum generator provided comprises a vacuum gauge.

16. A method according to claim 13 wherein the nose clamp provided comprises a substantially U-shaped clamp adapted to clamp and sealingly close the nares of a human's nose, which clamp is itself flexible and which clamp is flexibly connected to the mouthpiece.

17. A method of manufacturing a mouthpiece comprising:
a. constructing a mold adapted to be used in polypropylene injection molding processes, which mold comprises spaces to create:
i. a curved mouth cover adapted to sealingly fit over a human's mouth and lips;
ii. the mouth cover defining a nozzle opening adapted to be sealingly connected to and in fluid communication with a vacuum generator;
iii. the mouth cover further including a vacuum delivery hollow tongue depressor having at least one wall, the depressor integrally connected to the mouth cover and in fluid communication with the vacuum tube nozzle, the tongue depressor projecting away from the cover and the tongue depressor defining at least one hole; and
iv. a nose clamp having at least one living hinge and adapted to clamp the nares of a human's nose shut;
b. providing a source of heated, liquid polypropylene;
c. injecting the liquid polypropylene into the mold and unmolding the mouthpiece when appropriate; and
d. attaching a restraining band to the mouthpiece, which restraining band is adapted to hold the mouthpiece over the human's mouth and lips during use.
